(19) United States
(12) Patent Application Publication
(10) Pub. No.: US 2011/0009820 A1
(43) Pub. Date: Jan. 13, 2011

(54) Single-Use Syringe Allowing Aspiration

(76) Inventors: Phillip Oweb Byrne, Whickham (GB); Harry Raymond Ingham, Ponteland (GB); Penelope Rosemary Attridge, Middlesex (GB)

Correspondence Address: JACKIE JAY SCHWARTZ, 245 Fifth Avenue, Suite 1902, NEW YORK, NY, 10016, US

(21) Appl. No.: 12/673,616

(22) PCT Filed: Jun. 17, 2008
(86) PCT No.: PCT/GB08/50454

§ 371 Date: Feb. 16, 2010

(30) Foreign Application Priority Data
Aug. 17, 2007 (GB) 0716062.5

(51) Int. Cl. A61M 5/50

(52) U.S. Cl. 604/110

(57) ABSTRACT

A syringe (10) comprises a barrel (12) having an open first end (16) and a second end (18) having an opening (20) for passage of fluid therethrough. Furthermore, the syringe comprises a plunger (14) adapted to be slidably received in the first end and which has a shaft (24), and a head (26) for sliding and sealing engagement with an internal wall of the barrel. In addition, at least one locking member (30) engages the barrel and the shaft, having at least one first tine (32) for selectively engaging the plunger and at least one second tine (34) for selectively engaging an internal wall of the barrel. At least one locking member is adapted to restrict outward movement of the plunger relative to the barrel, but has sufficient flexibility to allow limited outward movement of the plunger relative to the barrel, subsequent to inward movement of the plunger following outward movement of the plunger relative to the barrel.

### TECHNICAL FIELD

The present invention relates to single use syringes and relates particularly to syringes which are adapted to prevent or at least hinder further use.

### BACKGROUND OF THE INVENTION

Disposable syringes are known in which a cylindrical barrel formed of transparent plastic material receives a piston, which is slidable within the barrel. A shaft, which may be of cruciform or other, e.g. circular, cross section extends from the piston to a plunger handle for enabling the piston to be displaced along the barrel in a first direction to cause injectable fluid, e.g. medicament or body fluid like blood, to be drawn into the barrel via an aperture at one end of the barrel, or in a second direction to cause the fluid to be expelled out of the aperture or to be injected into a patient via a needle.

Syringes of this type are generally sold as disposable items and are intended to be used only once to negate the risk of transmission of diseases between patients. However, such syringes suffer from the drawback that it is difficult to prevent such syringes from being re-used, which increases the risk of transmission of infection.

Numerous designs have been proposed for syringes, which are intended to negate or reduce the risk of the syringe being re-used. However, there are considerable challenges involved in designing a syringe that meets the following requirements:
(i) allowing aspiration or flashback of blood to check for correct location of the needle in a patient;
(ii) ability to deliver variable doses;
(iii) smooth operation;
(iv) simplicity of manufacture and use;
(v) ability to inject diluents (e.g. sterile water) into a vial of powdered/lyophilised drug, and/or allow agitation of vial or syringe content to assist powdered drug to go into solution.

EP0925083B1, for example, discloses a single-use syringe comprising a barrel with an internal annular groove at the proximal end and a further annular groove near the distal end but spaced from it. The plunger is formed with an integrally moulded barb-like flange adjacent the head, which flange bears resiliently against the barrel interior wall. The flange is able to move unrestricted in either direction along most of the barrel interior wall. However, the flange is only able to pass the annular grooves in one direction. The syringe is supplied with the plunger not fully depressed, so that the restrictor flange is on one side of one of the two grooves, facing the other groove. Liquid may be drawn up into the syringe until the flange encounters the other groove, which prevents the plunger being withdrawn completely from the barrel. Liquid may be discharged freely from the syringe by depressing the plunger fully into the barrel. In this position, the plunger is now prevented from being withdrawn again because the flange will not pass the grooves in the barrel.

This design suffers from a number of drawbacks. For example, there is often a slight jolt as the flange passes one of the grooves when an injection is being given, and this jolt is likely to be felt by a patient. Furthermore, because of the unrestricted movement of the plunger between the two grooves, the syringe could potentially be used again and again provided the plunger is never fully depressed. After unpacking a syringe, it is normal to cycle the plunger over a short distance to check that the plunger is free to move and, if it is not, to free it. There can sometimes be a degree of adhesion between the plunger head and the barrel due to the length of time of storage, or due to the effects of gamma sterilisation. This is particularly the case with plunger heads having silicone lubricant. During this movement it would be relatively easy to lock the plunger of the syringe by moving the flange past one of the grooves.

U.S. Pat. No. 5,000,737 discloses a syringe having a one-piece metal barbed restrictor element located between the plunger shaft and the cylindrical interior syringe barrel wall. The element has barbs facing towards the plunger for preventing movement of the plunger into the barrel, and barbs facing the barrel for preventing movement of the element out of the barrel. The restrictor element is initially located near the end of the barrel remote from the needle, and outward movement of the plunger to draw up liquid is permitted as the plunger can slide past the restrictor in this direction. Subsequent depression of the plunger to deliver liquid is permitted because the restrictor can move inwards relative to the barrel.

US2003/0060759 also utilises a one-piece metal barbed restrictor element mounted between the plunger shaft and the barrel interior wall, and it employs outwardly facing barbs to restrict the motion of the element relative to the barrel. In this design, however, the plunger shaft has a stepped form with a shoulder part way along it. The restrictor element has a spring tang, which acts against the barrel and forces it against the plunger shaft. The restrictor starts out at the end of the shaft remote from the needle and withdrawal of the plunger past the restrictor is permitted until an enlarged diameter portion of the shaft, near the plunger head, comes into engagement with the restrictor. At the same time, one end of the restrictor snaps behind the shoulder on the plunger. As a result, movement of the plunger in either direction with respect to the restrictor is prohibited. The plunger can be depressed, carrying the restrictor with it to the end of the barrel adjacent the needle, and then the plunger is incapable of further movement.

However, this syringe suffers from the disadvantage that it may be used repeatedly, provided the user does not draw up the plunger to the point where the restrictor snaps into place on the reduced diameter part of the plunger shaft. Furthermore, once the restrictor has locked into place, aspiration of a flash of blood is not possible.

U.S. Pat. No. 5,222,942 discloses a design based on a ratchet system. A collar is installed in an initial position between plunger shaft and barrel. The plunger shaft is formed with annular ratchet teeth, and corresponding teeth are formed on the collar. The ratchet does not permit the plunger to be moved outwards past the collar, so when the plunger is initially drawn back in order to draw up liquid, it carries the collar with it to the end of the barrel remote from the needle, where a formation on the barrel prevents the collar and plunger from being withdrawn completely from the barrel. The ratchet is such that the plunger may then be depressed past the collar to dispense liquid, and then the syringe is disabled. This design does not allow for aspiration of a flash of blood.

WO2005/058397A1 discloses a number of designs that are intended to allow for taking a flash of blood and possibly to allow for reconstitution of powdered drug. The designs described in WO2005/058397A1 are intended to be simple and inexpensive and to have a smooth action, which will not give rise to uncomfortable jolts when a patient is receiving an injection.

However, the designs described in WO2005/058397A1 work on the principle of providing for a small amount of “lost motion” between a restrictor element and the syringe plunger, which allows for unlimited repeated inward and outward movement over a distance which is just sufficient for the purpose of obtaining a flash of blood (or optionally for reconstitution of powdered drug). The distance may be carefully chosen so that it is sufficiently small to prevent, hinder or discourage repeated use of the syringe to deliver small doses of drug.

Fulfilment of all of the requirements for a non-reusable syringe in a single design is difficult, and there is normally a trade off between the complexity of the design and the number of these goals which are achieved by the design. One potential drawback with the designs described in WO2005/058397A1 is that they will allow drawing up and delivery of a volume of drug which is smaller than the usable volume of the syringe, and then drawing up and delivery of a further small dose or doses until the total volume of the syringe has been used. For example, a 5 ml syringe may be used to draw up 3 ml of liquid drug and then administer it. At this point the restrictor element has been moved a corresponding distance along the plunger or barrel, but there remains a distance corresponding to 2 ml of syringe volume over which the restrictor may move before the syringe is fully disabled. Further drawing up and delivery of a 2 ml dose of medicament is therefore possible before the syringe is fully disabled.

Preferred embodiments of the present invention seek to overcome at least some of the above disadvantages of the prior art.

### SUMMARY OF THE INVENTION

According to the present invention, there is provided a syringe comprising:
a barrel having an open first end and a second end having an opening for passage of fluid therethrough;
a plunger adapted to be slidably received in said first end and having a shaft, and a head for sliding and sealing engagement with an internal wall of said barrel; and
at least one locking member for engaging said barrel and said shaft, having at least one first tine for selectively engaging the plunger and at least one second tine for selectively engaging an internal wall of said barrel;

wherein at least one said locking member is adapted to restrict outward movement of the plunger relative to the barrel, but has sufficient flexibility to allow limited outward movement of the plunger relative to the barrel, subsequent to inward movement of the plunger following outward movement of the plunger relative to the barrel.

By providing a locking member for selectively engaging the internal wall of the barrel and the plunger to restrict outward movement, wherein the locking member has sufficient flexibility to allow limited repeated movement of the plunger relative to the barrel, this provides the advantage that the locking member only comprises a single part that is able to “lock” the plunger to the barrel after a single use and that allows repeated aspiration or flashback movements at any one point during operation. The invention therefore provides a simplified design requiring fewer parts and therefore significantly reduces the manufacturing costs of a single-use syringe.

The locking member may be adapted to be supported on said shaft and oriented in a first direction relative thereto, such that at least one said first tine and at least one said second tine face towards said first end.

At least one said first tine may be adapted to engage said shaft to restrict movement of the corresponding said locking member relative to the plunger when the plunger is moved into the barrel, and at least one said second tine may be adapted to engage said barrel to restrict movement of the locking member relative to the barrel when the plunger is moved out of the barrel.

At least one said locking member may be adapted to be received on said shaft and oriented in a second direction relative thereto, such that at least one said first tine and at least one said second tine face towards said second end.

At least one said first tine may be adapted to engage said shaft to restrict movement of the corresponding said locking member relative to the plunger when the plunger is moved out of the barrel, and at least one said second tine may be adapted to engage the internal wall of said barrel to restrict movement of the locking member relative to the barrel when the plunger is moved into the barrel.

At least part of said shaft may be provided with longitudinal extending ribs.

This provides the advantage of minimising the material that is necessary to provide a stiff and robust plunger shaft, therefore, minimising not only the weight of the plunger, but also reducing manufacturing costs.

At least one pair of said ribs may be arranged substantially orthogonally to each other.

At least one said rib may be arranged in a plane passing through a longitudinal axis of said shaft.

This provides the advantage of a very simple plunger design that is high in mechanical stability, low in weight and that provides support for a locking member.

At least one said locking member may be adapted to be received on at least one said rib.

At least one said locking member may have a said first tine and a said second tine arranged on opposite sides of said rib.

Said first tine and said second tine may be arranged on the same side of said rib.

This provides the advantage that the first and second tine can engage the barrel wall and plunger shaft on opposite sides of the rib providing a substantially symmetrical arrangement between the plunger shaft and the locking member. This therefore improves the robustness of the syringe.

At least one said locking member may further comprise a folded section adapted to engage said rib and to limit rotation of said locking member relative to said plunger.

This provides the advantage that the locking member is stable within the confines of the syringe barrel during operation.

At least one said first tine may be adapted to penetrate a wall of said shaft.

This provides the advantage that the locking member is able to sufficiently “friction-lock” to the plunger shaft when the plunger shaft is moved in the relevant direction, allowing the locking member to be moved with the plunger.

At least one said second tine may be adapted to penetrate a wall of said barrel.

This provides the advantage that the syringe is rendered unusable when a user tries to re-use the syringe after it has been “locked”.

The syringe may further comprise at least one flange adjacent a said first and/or second tine for limiting penetration of said tine through the corresponding wall of said barrel or plunger.

This provides the advantage of preventing injury to the operator when trying to re-use the syringe.

The barrel may further comprise at least one protrusion adjacent said first end to restrict removal of said locking member from said barrel.

This provides the advantage that the locking member can generally not be removed from the syringe, therefore, maintaining its functionality and improving durability and reliability of the syringe.

At least one said protrusion may be annular.

This provides the advantage that the locking member can engage the protrusion at any angular position of the plunger relative to the barrel.

At least one said locking member may be formed by bending an elongated member.

This provides the advantage that the locking member is easy to manufacture and therefore reduces manufacturing costs.

At least one said locking member may have at least one bend to provide sufficient flexibility to allow limited outward movement of the plunger relative to the barrel.

This provides the advantage that the limitation of movement of the plunger is adjustable through the number of bends provided at the locking member. This therefore allows syringes to be manufactured allowing different aspiration or flashback volume.

The feature which allows aspiration of blood may be operative at any position of the plunger so that the feature can be used whatever volume of injectable material is contained in the syringe and also may be used to check the needle position in a patient prior to drawing a blood sample, i.e. when the syringe is substantially empty and the plunger is fully inserted into the barrel. Therefore, preferably the said limited repeated cycles of distal and proximal movement referred to above are permitted at substantially every relative position of the plunger and barrel over the usable range.

The distance over which repeated movement is possible is, desirably, sufficient to aspirate a small volume of blood from a patient so as to check the position of the needle.

Preferred embodiments of the present invention will now be described, by way of example only and not in any limitative sense, with reference to the accompanying drawings in which:

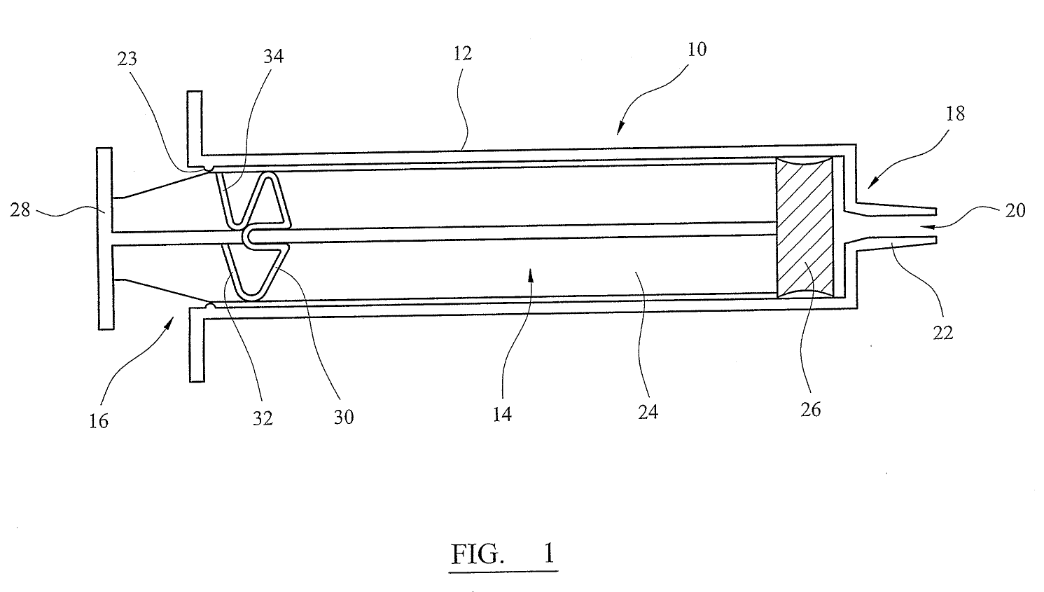

FIG. 1 shows a sectional view of a syringe according to a first embodiment of the invention;

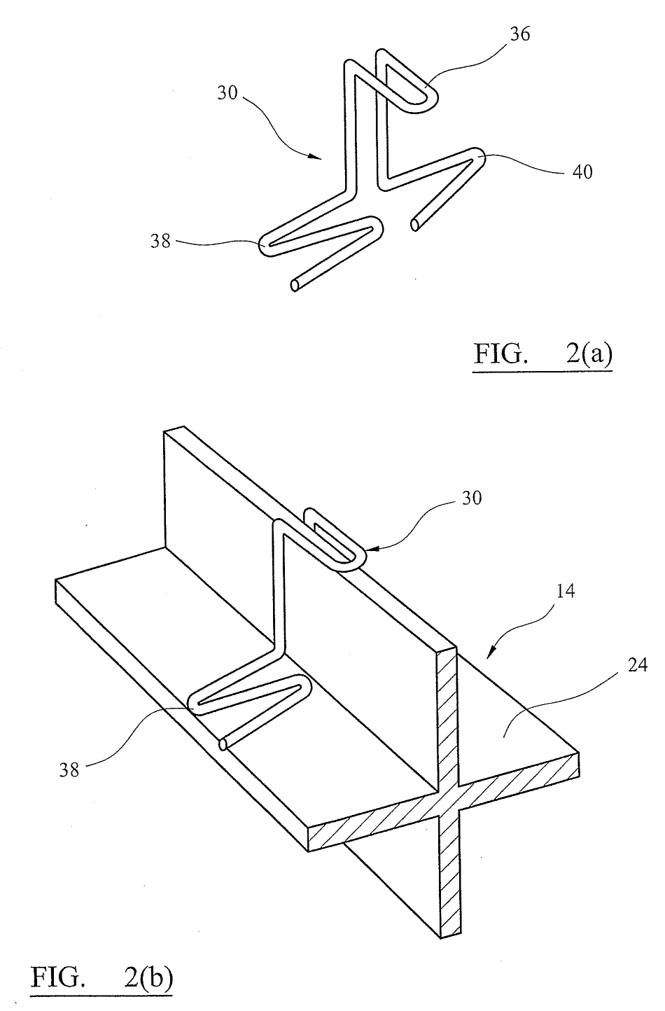

FIG. 2a is a perspective view of a locking member of the syringe of FIG. 1;

FIG. 2b is a perspective view of a syringe plunger shaft with the locking member of FIG. 2a;

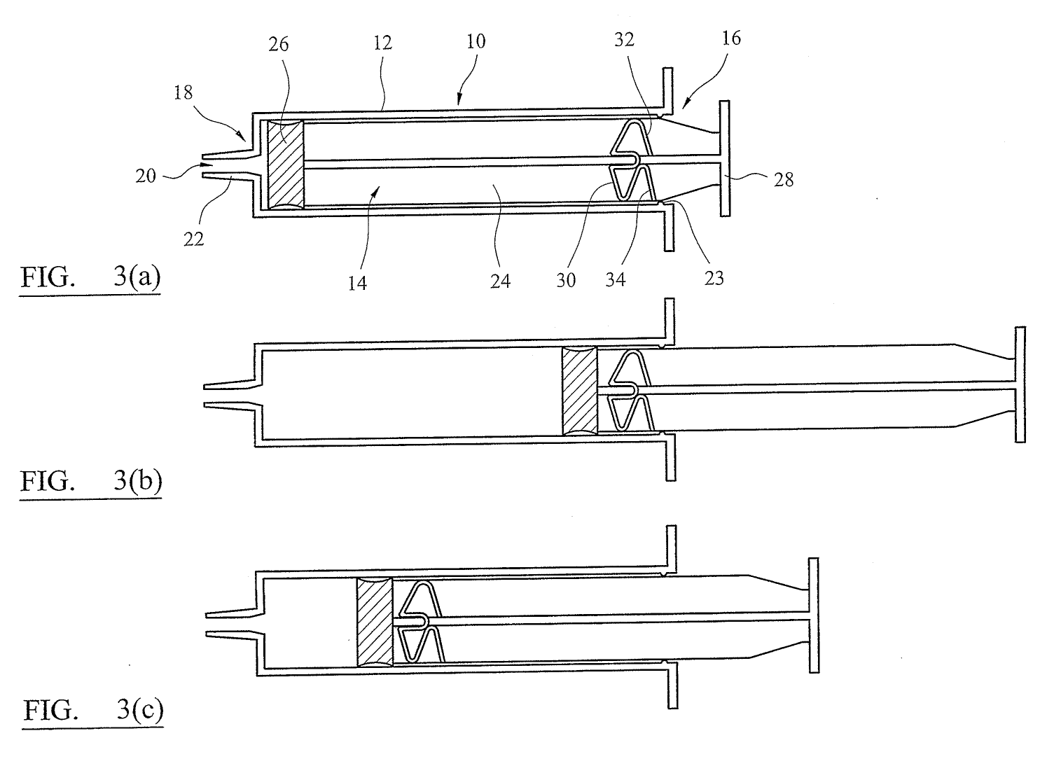

FIG. 3 shows a sectional view of the syringe of FIG. 1 at three stages during operation, (a) in the starting position, (b) with the plunger fully withdrawn and (c) with the plunger partly depressed during operation;

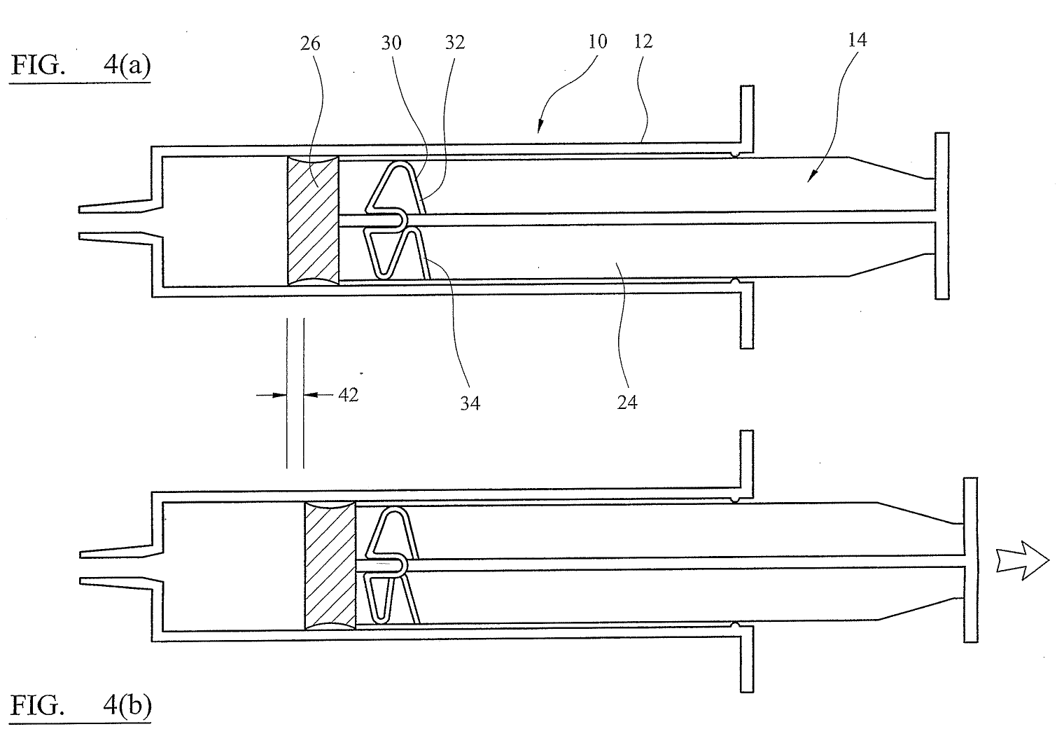

FIG. 4 shows a sectional view of the syringe of FIG. 1 at two stages during operation, (a) with the plunger partly depressed subsequent to withdrawal of the plunger and (b), with the plunger pulled back due to sufficient flexibility of the locking member;

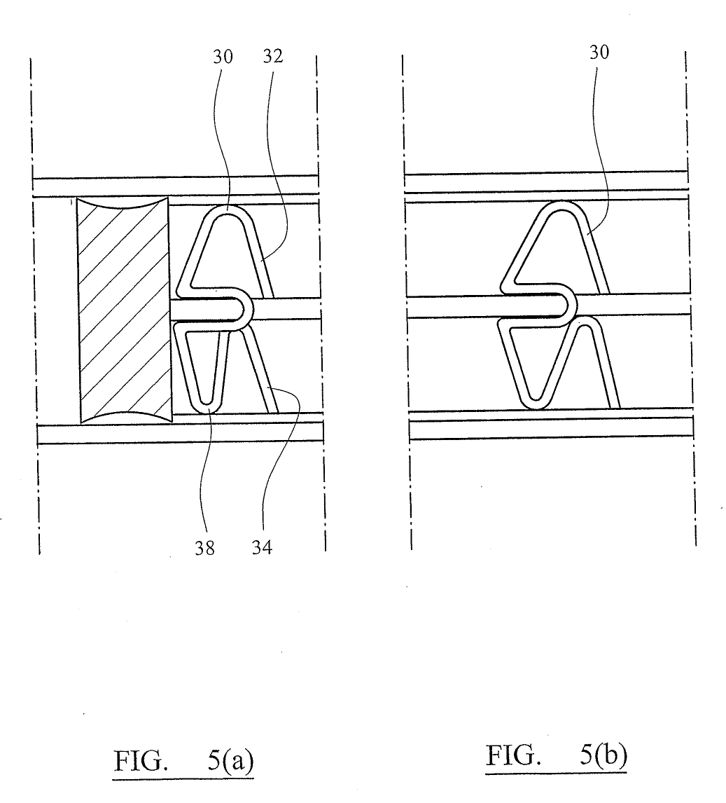

FIG. 5 shows a close-up of a partial sectional view of the syringe of FIG. 1 with the assembled locking member (a) under compression by the plunger and (b) in a relaxed position;

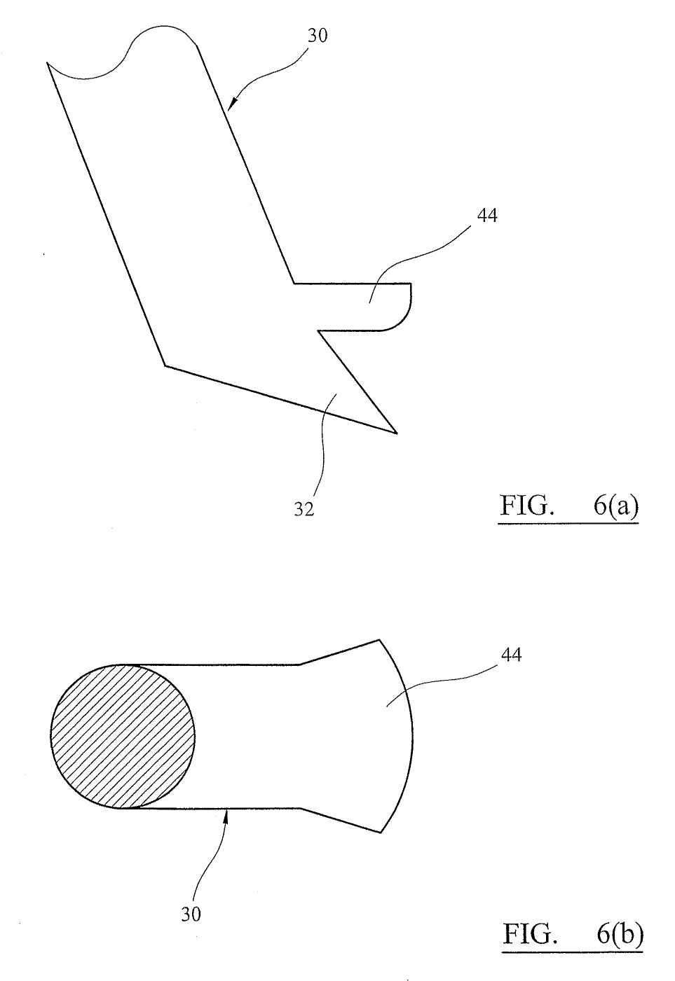

FIG. 6 shows a partial view of a tine having a flange from the side (a) and from the top (b);

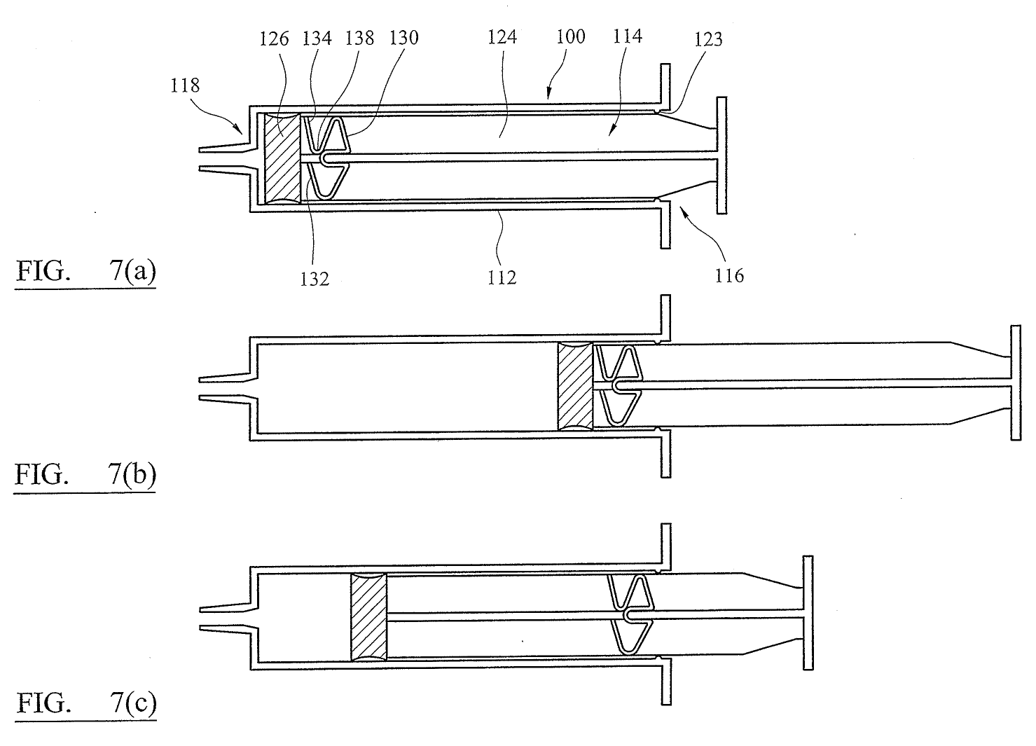

FIG. 7 shows a sectional view of a syringe according to a second embodiment of the invention at three stages during operation, (a) in the starting position, (b) with the plunger fully withdrawn and (c) with the plunger partly depressed subsequent to the withdrawal of the plunger;

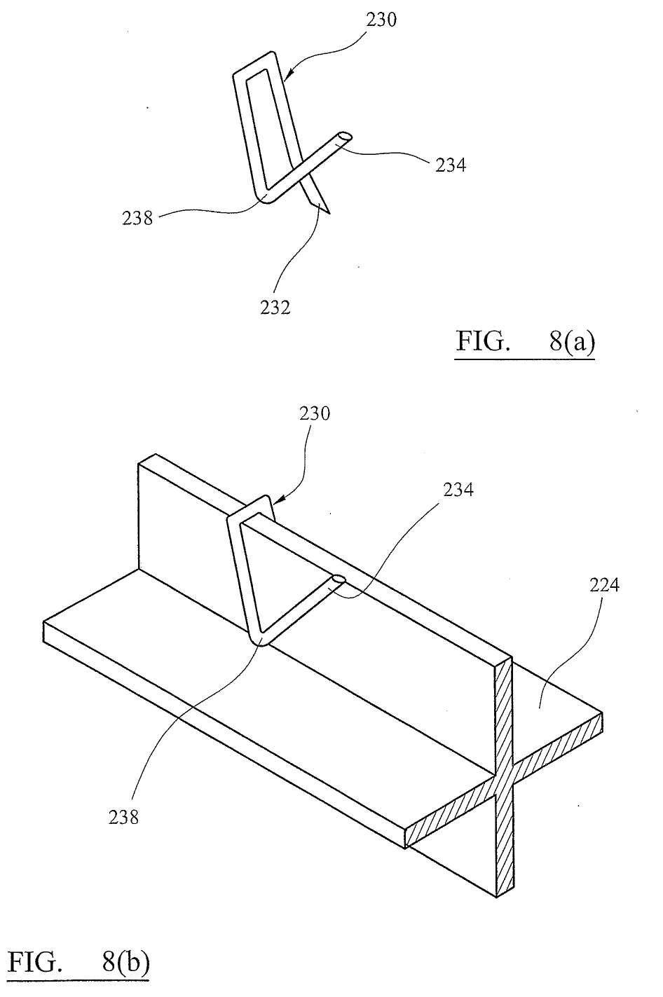

FIG. 8a is a perspective view of a locking member according to a third embodiment of the invention;

FIG. 8b is a perspective view of a syringe plunger shaft with the locking member of FIG. 8a;

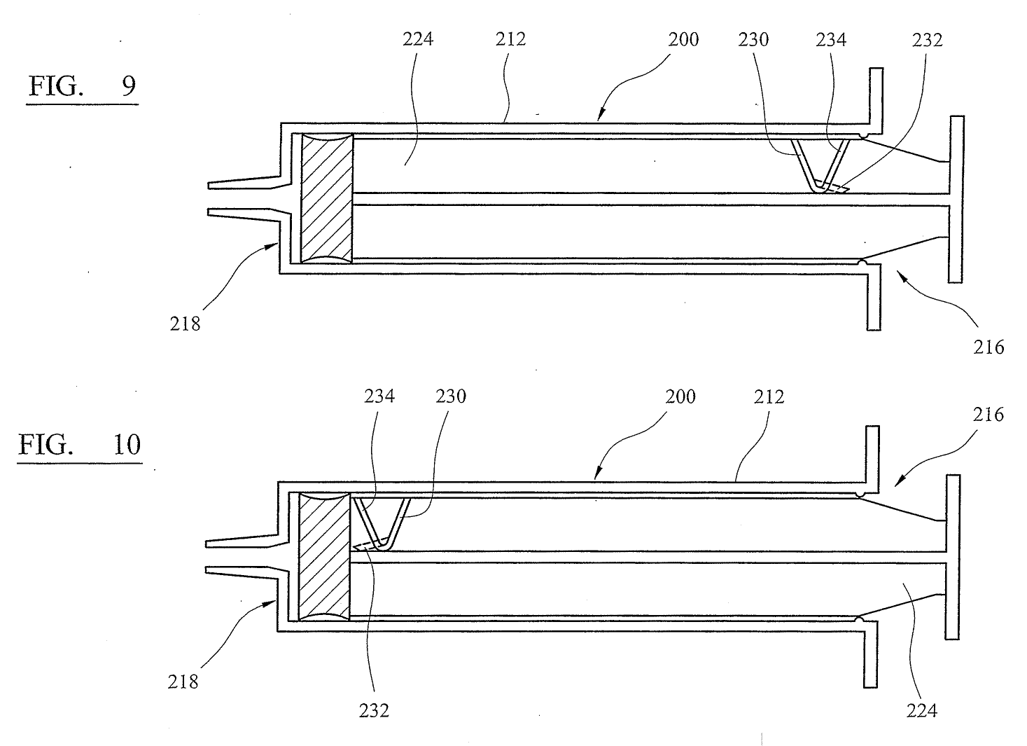

FIG. 9 shows a sectional view of a syringe according to the third embodiment of the invention with the locking member located in a first direction; and

FIG. 10 shows a sectional view of a syringe according to the third embodiment of the invention with the locking member located in a second direction.

### DETAILED DESCRIPTION OF EMBODIMENTS

### First embodiment

Referring to FIG. 1, a first embodiment is based on a standard syringe 10, normally constructed from plastic material, comprising a barrel 12 and a plunger 14. The barrel 12 has an open first end 16 and a generally closed second end 18 having an opening 20 that allows gases or liquids to pass and a nipple-like protuberance 22 or, for example, a Luer connector, allowing needles or tubing to be connected. Preferably, the barrel 12 has a tubular cross-section, but could also be of any other suitable cross-section. The barrel 12 further comprises, for example, an annular protrusion 23 that is located adjacent to the open end 16 preventing the removal of the plunger 14.

The plunger 14 comprises a plunger shaft 24, which sealingly engages the interior wall of the barrel 12 and is usually of cruciform construction, and a flexible bung 26, which is affixed to the distal end of the plunger shaft 24 forming a slidable seal. A handle 28 is incorporated on the proximal end of the plunger shaft 24 to facilitate forward and backward movement of the plunger 14.

A locking member 30 in the form of a small jockey is constructed from a spring-like material, e.g. steel or plastic, and fixed to one of the ribs of the cruciform plunger shaft 24. The locking member 30 may be made from an elongated wire, having a sharpened first tine 32 and a sharpened second tine 34 on respective ends of the wire, and which is bent in such a way that the first tine 32 is able to engage the plunger shaft 24 and the second tine 34 is able to engage the interior wall of the barrel 12. In this first embodiment of the invention, the locking member 30 is oriented in a first direction relative to the plunger shaft 24, as shown in FIGS. 1 to 5, where both tines face away from the needle of the syringe towards the first end 16 of the barrel 12.

Referring to FIGS. 2a and 2b, the locking member 30 may comprise a folded section 36, which sits on top of a rib of the plunger shaft 24 in order to provide stability to the locking member 30 during operation of the syringe 10. In particular, the folded section 36 prevents the locking member 30 from rotating about an axis perpendicular to the longitudinal wall of the rib. Also, the locking member 30 may have at least one additional bend 38 to provide a predetermined flexibility to the locking member, in order to allow repeated aspiration or flashback at any one point during operation.

The operation of the syringe 10 according to the first embodiment is now described with reference to FIGS. 3 (a), (b) and (c). The syringe 10 may be delivered in assembled form with the plunger 14 and the locking member 30 already in starting position 3(a). Here, the plunger 14 is fully depressed inside the barrel 12 and the locking member 30 is located on the plunger shaft 24 at the first end 16 and inside the barrel 12. The first and second tine 32, 34 face towards the first end 16 and the second tine 34 abuts the annular protrusion 23. On the other hand, the syringe 10 may be delivered in disassembled form, wherein the operator simply attaches the locking member 30 to the proximal end of one of the ribs of the plunger shaft 24 and pushes the plunger 14 inside the barrel by carefully negotiating the locking member 30 past the annular protrusion 23.

Once in starting position, as shown in FIG. 3 (a), the operator may attach a needle or tubing to the nipple-like protuberance 22 and the syringe is ready for use. The needle may now be placed inside a vein of a patient to withdraw blood or may be used to fill the barrel 12 with a medicament, which might be administered to the patient. During operation, the operator withdraws the plunger 14 moving the flexible bung 26 towards the first end 16 of the barrel 12, thus filling the syringe 10 with blood or medicament. The resilience of the locking member 30 in combination with its first and second tine 32, 34 is such that during withdrawal of the plunger 14, the locking member 30 remains in place as shown in FIG. 3(b). When the plunger 14 is then depressed to expel the contents of the syringe 10, the first tine 32 engages and digs into the rib of the plunger shaft 24, the second tine 34 is positioned such that it slides against the interior wall of the barrel 12. This allows the locking member 30 to travel with the plunger 14 towards the second end 18 of the barrel 12 as shown in FIG. 3 (c).

Any attempt to withdraw the plunger 14 and its associated bung 26, after the plunger 14 has been withdrawn and moved back inside the barrel 12, will push the plunger 14 against the locking member 30 and engage the second tine 34 with the interior wall of the barrel 12, preventing the locking member 30 from moving towards the first end 16 of the barrel 12. Further application of a withdrawing force results in compressing of the additional bends 38 of the locking member. In case a predetermined withdrawing force is exceeded, the second tine 34 may destroy the barrel 12 of the syringe 10 by cutting through the barrel wall.

FIG. 4(a) shows the plunger 14 in a partly depressed position after the plunger 14 has been withdrawn from the starting position shown in FIG. 3(a) and pushed back into the barrel 12 and without any withdrawing force acting on the plunger 14. FIG. 4 (b) shows the locking member 30 in a compressed form during further application of a withdrawing force. This allows repeated aspiration or flashback denoted by the plunger movement 42. Aspiration or flashback is primarily determined by the flexibility of the locking member 30. An increased number of additional bends 38, different designs of bends, and/or a ‘softer’ material of the locking member 30 may therefore increase the aspiration or flashback movement 42 of the syringe 10. On the other hand, a decreased number of bends 38 and/or a ‘harder’ material of the locking member 30 may decrease the aspiration or flashback movement 42 of the syringe 10.

FIGS. 5 (a) and (b), show respectively a close-up of a sectional view of the locking member 30 during operation when (a) compressed by a withdrawing force of the plunger 14 and (b) in a relaxed state without any additional withdrawing force from the plunger 14. Both first tine 32 and second tine 34 are engaged with the plunger shaft 24 and the interior wall of the barrel 12, respectively, therefore restricting the movement of the plunger 14 towards the first end 16 of the barrel 12.

Referring to FIGS. 6 (a) and 6 (b), a flange 44 may be located adjacent to the first tine 32 and/or adjacent to the second tine 34 to prevent the first and/or second tine 32, 34 from completely penetrating the wall of the barrel 12. This prevents the operator from being injured on a tine that protrudes from the wall of the barrel 12. In particular, the flange 44 may be located at a predetermined distance from the first and/or second tine 32, 34 that is equal or less than the thickness of the interior wall of the barrel 12. Hence, the first and/or second tine 32, 34 will not be able to travel through and onto the outside of the barrel wall, therefore, minimizing the risk of injuries to the operator. The application of extreme withdrawal forces will cause tine 34 to plough a groove into the wall of the barrel to destroy the integrity of the syringe.

### Second Embodiment

A second embodiment of the invention will now be described with reference to FIGS. 7 (a), 7 (b) and 7 (c). Here, the locking member 130 is attached to the plunger shaft 124 oriented in a second direction. The syringe 100 may be delivered in assembled form with the plunger 114 and the locking member 130 already in starting position as shown in FIG. 7 (a). Here, the plunger 114 is fully depressed inside the barrel 112 and the locking member 130 is located on the plunger shaft 124 at the second end 118 inside the barrel 112, with the first tine 132 and the second tine 134 facing towards the second end 118. On the other hand, the syringe 100 may be delivered in disassembled form, wherein the operator simply attaches the locking member 130 to the distal end of one of the ribs of the plunger shaft 124 and with the aid of a suitable tool, which will be familiar to persons skilled in the art, pushes the plunger 114 inside the barrel 112 by carefully negotiating the locking member 130 past the annular protrusion 123 to the second end 118 of the barrel 112.

During operation, the operator withdraws the plunger 114 moving the flexible bung 126 and the locking member 130 to the first end 116 of the barrel 112, thus filling the barrel 112 with blood or a medicament. During movement of the plunger 114, second tine 134 slidably moves past the interior wall of the barrel 112, wherein first tine 132 engages with the flexible bung 126, preventing movement of the locking member 130 relative to the plunger 114. As shown in FIG. 7 (b), the locking member 130 is now located at the first end 116 of the barrel 112 abutting the annular protrusion 123. When the plunger 114 is depressed to expel the content of the syringe 100, the second tine 134 engages and digs into the interior wall of the barrel 112, therefore, preventing any movement of the locking member 130 relative to the barrel 112, as shown in FIG. 7 (b), As shown in FIG. 7 (c), the first tine 132 is positioned such that it slides against the rib of the plunger shaft 124 allowing the plunger 114 to travel towards the second end 118 of the barrel 112.

Any attempt to withdraw the plunger 114 and its associated bung 126, after the plunger 114 has been withdrawn and moved back inside the barrel 112, will engage the locking member 130 with the annular protrusion 123 of the barrel 112, the first tine 132 with the plunger shaft 124 and the second tine 134 with the interior wall of the barrel 112 preventing any movement of the plunger 114 relative to the barrel. Further application of a withdrawing force may result in the bends 138 of the locking member being compressed, allowing repeated aspiration or flashback. In the event that a predetermined withdrawing force is exceeded, the second tine 134 may destroy the barrel 112 by puncturing the wall. With the locking member at the end 116, the plunger can again be moved towards end 118. Tine 134 engages the internal wall of the syringe barrel 112 locking the member 130 in place at the position 116. Tine 132 is so positioned that the plunger shaft 114 is free to move towards the end 118. Any additional movement towards the end 116 causes tine 116 to engage the plunger shaft stopping any further withdrawal. Compression of member 130 allows flashback.

It will be appreciated by persons skilled in the art that an increased number of additional bends 138 and/or a ‘softer’ material of the locking member 130 may increase the aspiration or flashback movement of the syringe 100, and that a decreased number of bends 138 and/or a ‘harder’ material of the locking member 130 may decrease the aspiration or flashback movement of the syringe 100.

The locking member 130 may also comprise a flange adjacent to the first and/or second tine 132, 134 as described in the first embodiment to prevent the operator from being injured by exposed tines.

### Third Embodiment

A third embodiment of the invention will now be described with reference to FIGS. 8 (a), 8 (b), 9 and 10. The third embodiment comprises a syringe 200 similar to the first embodiment, having a locking member 230, for example in the form of a small jockey, that is constructed from a spring-like material, e.g. steel or plastic, and fixed to one of the ribs of the cruciform plunger shaft 224. The locking member 230 may be made from an elongated wire, having a sharpened first tine 232 and a sharpened second tine 234 on respective ends of the wire, which is bend in such a way that the first tine 232 is able to engage the plunger shaft 224 and the second tine 234 is able to engage the interior wall of the barrel 212 of the syringe 200. As shown in FIG. 9, the locking member 230 may be oriented in a first direction relative to the plunger shaft 224, where both tines face away from the needle of the syringe towards the first end 216 of the barrel 212. On the other hand, as shown in FIG. 10, the locking member 230 may be oriented in a second direction relative to the plunger shaft 224, where both tines 232, 234 face towards the second end 218 of the barrel 212.

Referring to FIGS. 8a and 8b, the locking member 230 may only comprise a single additional bend 238 on one side of the rib of the plunger shaft 214 to provide a predetermined flexibility to the locking member, therefore allowing repeated aspiration or flashback at any one point during operation.

The operation of the syringe according to the third embodiment, with the locking member 230 in the first direction, resembles with the operation described for the syringe according to the first embodiment and, with the locking member 230 in the second direction, resembles with the operation described for the syringe according to the second embodiment.

It will be appreciated by persons skilled in the art that the above embodiments have been described by way of example only, and not in any limitative sense, and that various alterations and modifications are possible without departure from the scope of the invention as defined by appended claims.

1. A syringe comprising:
a barrel having an open first end and a second end having an opening for passage of fluid therethrough;
a plunger adapted to be slidably received in said first end and having a shaft, and a head for sliding and sealing engagement with an internal wall of said barrel; and
at least one locking member for engaging said barrel and said shaft, having at least one first tine for selectively engaging the plunger and at least one second tine for selectively engaging an internal wall of said barrel;
wherein at least one said locking member is adapted to restrict outward movement of the plunger relative to the barrel, but has sufficient flexibility to allow limited outward movement of the plunger relative to the barrel, subsequent to inward movement of the plunger following outward movement of the plunger relative to the barrel.

2. A syringe according to claim 1, wherein said locking member is adapted to be supported on said shaft and oriented in a first direction relative thereto, such that at least one said first tine and at least one said second tine face towards said first end.

3. A syringe according to claim 2, wherein
at least one said first tine is adapted to engage said plunger to restrict movement of the corresponding said locking member relative to the plunger when the plunger is moved into the barrel, and
at least one said second tine is adapted to engage said barrel to restrict movement of the locking member relative to the barrel when the plunger is moved out of the barrel.

4. A syringe according to any one of the preceding claims, wherein at least one said locking member is adapted to be received on said shaft and oriented in a second direction relative thereto, such that at least one said first tine and at least one said second tine face towards said second end.

5. A syringe according to claim 4, wherein
at least one said first tine is adapted to engage said shaft to restrict movement of the corresponding said locking member relative to the plunger when the plunger is moved out of the barrel, and
at least one said second tine is adapted to engage said internal wall of said barrel to restrict movement of the locking member relative to the barrel when the plunger is moved into the barrel.

6. A syringe according to any one of the preceding claims, wherein at least part of said shaft is provided with longitudinally extending ribs.

7. A syringe according to claim 6, wherein at least one pair of said ribs are arranged substantially orthogonally to each other.

8. A syringe according to claim 6 or 7, wherein at least one said rib is arranged in a plane passing through a longitudinal axis of said shaft.

9. A syringe according to any one of claims 6 to 8, wherein at least one said locking member is adapted to be received on at least one said rib and has a said first tine and a said second tine arranged on opposite sides of said rib.

10. A syringe according to claim 9, wherein at least one said locking member further comprises a folded section adapted to engage said rib and to limit rotation of said locking member relative to said plunger.

11. A syringe according to any one of the preceding claims, wherein at least one said first tine is adapted to penetrate a wall of said shaft.

12. A syringe according to any one of the preceding claims, wherein at least one said second tine is adapted to penetrate a wall of said barrel.

13. A syringe according to claim 11 or 12, further comprising at least one flange adjacent a said first and/or second tine for limiting penetration of said tine through the corresponding wall of said barrel or plunger.

14. A syringe according to any one of the preceding claims, wherein said barrel further comprises at least one protrusion adjacent said first end to restrict removal of said locking member from said barrel.

15. A syringe according to claim 14, wherein at least one said protrusion is annular.

16. A syringe according to any one of the preceding claims, wherein at least one said locking member is formed by bending an elongated member.

17. A syringe according to claim 16, wherein at least one said locking member has at least one bend to provide sufficient flexibility to allow limited outward movement of the plunger relative to the barrel.
